(19) United States
(12) Patent Application Publication
(10) Pub. No.: US 2026/0060518 A1
(43) Pub. Date: Mar. 5, 2026

(54) ENDOSCOPE LIGHT SOURCE SYNCHRONIZATION SYSTEM

(71) Applicant: HUKUI BIOTECHNOLOGY CO., LTD., Taipei City (TW)

(72) Inventors: SHIH-CHE LO, Taipei City (TW); YI-MING CHU, Taipei City (TW); MING-KUN CHAN, Taipei City (TW)

(73) Assignee: HUKUI BIOTECHNOLOGY CO., LTD., Taipei City (TW)

(21) Appl. No.: 19/208,627

(22) Filed: May 15, 2025

Related U.S. Application Data
(60) Provisional application No. 63/690,813, filed on Sep. 5, 2024.

(30) Foreign Application Priority Data
Feb. 27, 2025 (TW) 114107546

(51) Int. Cl. A61B 1/06; A61B 1/00; A61B 1/07; H04N 23/72; H04N 23/73; H04N 23/74; H04N 23/76

(52) U.S. Cl. CPC A61B 1/0655; A61B 1/00045; A61B 1/0638; A61B 1/0646; A61B 1/0684; A61B 1/07; H04N 23/72; H04N 23/73; H04N 23/74; H04N 23/76

(57) ABSTRACT

The present application provides an endoscope light source synchronization system adapted to connect a display device, an endoscope device and a light source device, where the endoscope device includes an image sensor and the light source device is used to emit at least two kinds of light sources. The endoscope light source synchronization system includes an image signal processor and a timing control module, where the timing control module uses a synchronization signal to synchronously control the light source device and the image sensor, and synchronously controls the light source device to switch and activate the light sources according to different light sources required by the image sensor in each frame, so that the image sensor generates different image signals corresponding to different light sources, and the image signal processor simultaneously displays several corresponding image screens on the display device according to the image signals.

### CROSS-REFERENCE TO RELATED APPLICATION

This non-provisional application claims priority under 35 U.S.C. § 119(e) on U.S. provisional Patent Application No(s).63/690,813 filed on Sep. 5, 2024, the entire contents of which are hereby incorporated by reference.

This non-provisional application claims priority under 35 U.S.C. § 119(a) on Patent Application No(s). 114107546 filed in Taiwan, R.O.C. on Feb. 27, 2025, the entire contents of which are hereby incorporated by reference.

### BACKGROUND OF THE INVENTION

### 1. Field of the Invention

The present disclosure relates to an endoscope system, and in particular to an endoscope light source synchronization system that makes an image and a light source operate synchronously.

### 2. Description of the Related Art

The incidence of esophageal cancer and head and neck cancer has been increasing year by year. When symptoms occur and diagnosis is made, a physician will firstly conduct an endoscopic examination with a white light source, and then switch the white light source to a narrow band imaging light source for confirmation when a suspected sign is detected. Therefore, at each moment, an image with only one kind of light source is available for the physician to interpret.

### BRIEF SUMMARY OF THE INVENTION

When conducting endoscopic examination, a physician cannot accurately obtain a narrow band imaging light image corresponding to a white light image for a position with a suspected sign due to the fact that an endoscope moves during operation to cause the relative positions of images shot under different light sources before and after to be different, so that the physician makes a biased judgment about the sign.

The applicant develops an endoscope light source synchronization system with all his might to devote himself to the study, which can achieve a purpose of synchronous operation of an image and a light source.

A secondary purpose of the present disclosure is to provide an endoscope light source synchronization system, where several image screens generated by the endoscope light source synchronization system after switching different light sources can look like a continuous screen in human eyes.

The present disclosure provides an endoscope light source synchronization system, adapted to connect a display device, an endoscope device and a light source device, where the endoscope device includes an image sensor, the light source device is used to emit at least two kinds of light sources and irradiate a part to be detected, and the endoscope light source synchronization system includes: an image signal processor, electrically connected to the display device and the image sensor; and a timing control module, electrically connected to the image sensor and the light source device, where the timing control module uses a synchronization signal to synchronously control the light source device and the image sensor, and synchronously controls the light source device to switch and activate the light sources according to different light sources required by the image sensor in each frame, so that the image sensor generates different image signals corresponding to different light sources; and the image signal processor simultaneously displays several corresponding image screens on the display device according to the image signals.

In an embodiment, the light source device includes several light-emitting diodes and the several light-emitting diodes are used to emit light sources with different colors and provided in the endoscope device.

In an embodiment, the image signal processor is provided in a host body; an outer surface of the host body is provided with a first connecting hole and a second connecting hole to be electrically connected to the image signal processor respectively; the first connecting hole is used to connect the display device; and the second connecting hole is used to connect the endoscope device.

In an embodiment, the light source device is provided in the host body and is a combination of a halogen lamp and several filters with different colors; and the light source emitted by the light source device is transmitted to the endoscope device via an optical fiber.

In an embodiment, the light sources emitted by the light source device are at least two kinds of light sources of white light, structured light, infrared light, ultraviolet light, fluorescent excitation light, and narrow band imaging light.

In an embodiment, the timing control module is provided in the endoscope device or the host body.

In an embodiment, the image sensor sets a corresponding exposure parameter according to different light sources required in each frame.

In an embodiment, the exposure parameters include a shutter time and a gain.

In an embodiment, the endoscope light source synchronization system of the present disclosure further includes an identification module electrically connected to the image signal processor, where the light sources emitted by the light source device include narrow band imaging light; the identification module performs image identification on image screens generated by the narrow band imaging light to identify whether the part to be detected has a sign or not, and transmits a result to the image signal processor; and the image signal processor controls the display device to display the result.

In an embodiment, the endoscope light source synchronization system of the present disclosure further includes a modeling module electrically connected to the image signal processor, where the light sources emitted by the light source device include structured light; the modeling module generates a three-dimensional image according to an image signal generated by the structured light and transmits the three-dimensional image to the image signal processor; and the image signal processor controls the display device to display the three-dimensional image.

In an embodiment, the endoscope light source synchronization system of the present disclosure further includes an optical filter, where the optical filter is provided in front of the image sensor to filter out some wavelengths of light rays.

In an embodiment, the endoscope light source synchronization system of the present disclosure further includes an image merging module, where the image merging module is electrically connected to the image signal processor and is used to merge image screens generated under two different light sources into a fused image.

To sum up, the endoscope light source synchronization system of the present disclosure can synchronously control the image sensor to start operating when the light source device is controlled to start operating, and a set light source irradiates the part to be detected, which can achieve an effect of avoiding that the image screens generated under different light sources skew during switching. Furthermore, in an asynchronous processing way of switching light sources and then shooting images, when the image screens generated under different light sources are displayed on a screen, multiple image screens cannot exist in human vision synchronously, that is, the physician can only view the image screens generated by one of the light sources in the same period. However, when the image screens generated in the present disclosure are displayed on the corresponding screens, respectively, because of the persistence of human vision, the image screens look like a continuous screen when the physician views the image screens on each screen. Therefore, the physician can view the image screens generated by several different light sources respectively in the same period, which achieves an effect of improving the accuracy of the detection and analysis of the physician of whether the part to be detected has the sign or not.

### BRIEF DESCRIPTION OF THE DRAWINGS

FIG. 1 is a block diagram of a first embodiment of an endoscope light source synchronization system of the present disclosure.

FIG. 2 is a three-dimensional diagram of a host body of an endoscope light source synchronization system of the present disclosure.

FIG. 3 is a block diagram of a second embodiment of an endoscope light source synchronization system of the present disclosure.

FIG. 4 is a block diagram of a third embodiment of an endoscope light source synchronization system of the present disclosure.

FIG. 5 is a block diagram of a fourth embodiment of an endoscope light source synchronization system of the present disclosure.

FIG. 6 is a block diagram of a fifth embodiment of an endoscope light source synchronization system of the present disclosure.

FIG. 7 is a block diagram of a sixth embodiment of an endoscope light source synchronization system of the present disclosure.

FIG. 8 is a block diagram of a seventh embodiment of an endoscope light source synchronization system of the present disclosure.

FIG. 9 is a block diagram of an eighth embodiment of an endoscope light source synchronization system of the present disclosure.

FIG. 10 is a timing diagram of a specific embodiment of an endoscope light source synchronization system of the present disclosure.

FIG. 11 is a timing diagram of another specific embodiment of an endoscope light source synchronization system of the present disclosure.

FIG. 12 is a timing diagram of yet another specific embodiment of an endoscope light source synchronization system of the present disclosure.

FIG. 13 is a timing diagram of still another specific embodiment of an endoscope light source synchronization system of the present disclosure.

### DETAILED DESCRIPTION OF THE INVENTION

In order to fully understand the purposes, features and efficacy of the present disclosure, the present disclosure is described below in detail by way of the following specific embodiments in conjunction with the accompanying drawings, as follows:

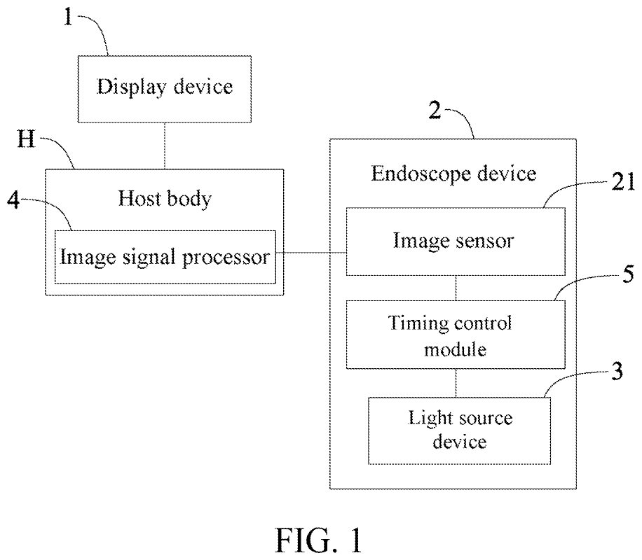

Referring to FIG. 1, FIG. 1 is a block diagram of a first embodiment of an endoscope light source synchronization system of the present disclosure. The endoscope light source synchronization system is adapted to connect a display device 1, an endoscope device 2, and a light source device 3, and includes an image signal processor 4 and a timing control module 5, where the image signal processor 4 is electrically connected to the display device 1 and the endoscope device 2; and the timing control module 5 is electrically connected to the endoscope device 2 and the light source device 3.

In a specific embodiment, a number of the display device 1 may be one, so that several image screens are displayed on a screen simultaneously. Alternatively, the number of the display devices 1 may be more than one, so that several image screens are distributed on several screens and displayed simultaneously. In this embodiment, the display device 1 may be an LCD screen.

In a specific embodiment, the endoscope device 2 is used to examine a human body cavity, and includes an image sensor 21, where the image sensor 21 is used to sense and receive an image signal generated by a part to be detected under the irradiation of the light source device 3. The part to be detected is a position where a human body is suspected to have a lesion. For example, it may be the esophagus or an interior of the neck, but is not limited thereto.

In a specific embodiment, the light source device 3 is used to emit at least two kinds of light sources and irradiate the part to be detected. In this embodiment, the light sources emitted by the light source device 3 are at least two kinds of light sources of white light, structured light, infrared light, ultraviolet light, fluorescent excitation light, and narrow band imaging (NBI) light. The light source device 3 may include several light-emitting diodes, and the several light-emitting diodes are used to emit light sources with different colors and preferably can be provided in the endoscope device 2. For example, but without limitations, the light source device 3 can be controlled by a switch controller (for example, an open collector) to achieve an effect of reducing the energy consumption of the endoscope device.

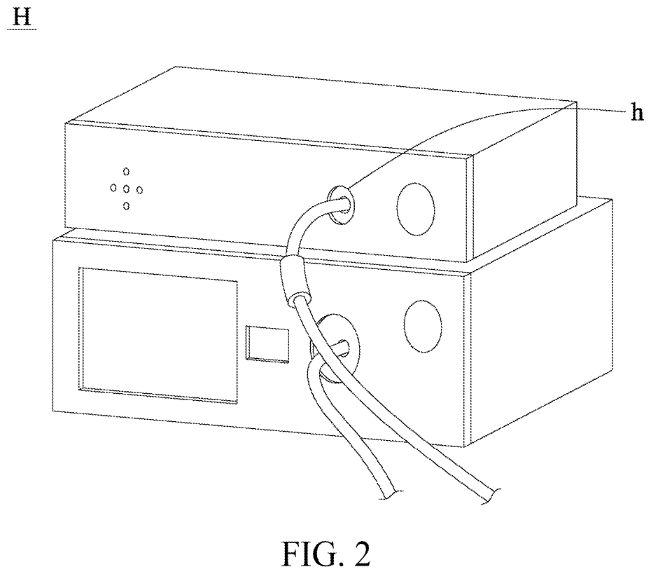

Also, referring to FIG. 2, FIG. 2 is a three-dimensional diagram of a host body of an endoscope light source synchronization system of the present disclosure. In a specific embodiment, the image signal processor 4 can send out a synchronization signal (Frame Synchronization Pulse, F_sync) that is used to control the synchronous operation of the image sensor 21 and the light source device 3, and can be provided in a host body H. An outer surface of the host body H is provided with a first connecting hole (not shown) and a second connecting hole h to be electrically connected to the image signal processor 4 respectively, where the first connecting hole is used to connect the display device 1; and the second connecting hole h is used to connect the endoscope device 2. In this embodiment, the host body H may be an endoscope image workstation, and the image signal processor 4 may have image processing functions such as color correction, conversion, gamma correction, and be used to output an image frame. In this embodiment, the image frame may include information such as images and light sources. It is worth mentioning that a signal source of this synchronization signal can also be sent out by the image sensor 21, but not by the image signal processor 4.

In a specific embodiment, the timing control module 5 preferably may be a microcontroller unit (MCU). Specifically, the timing control module 5 uses the synchronization signal to synchronously control the light source device 3 and the image sensor 21, and synchronously controls the light source device 3 to switch and activate the light sources according to different light sources required by the image sensor 21 in each frame, so that the image sensor 21 generates different image signals corresponding to different light sources. Subsequently, the image signal processor 4 simultaneously displays several corresponding image screens on the display device 1 according to the image signals. In this embodiment, the timing control module 5 can be provided in the endoscope device 2. The switching time of the light sources may be shorter than the time of persistence of human vision (e.g., 1/16 second).

Preferably, the image sensor 21 can set a corresponding exposure parameter, e.g., a shutter time and a gain, according to different light sources required in each frame. By way of example, when the light source device 3 switches to white light as a light source, the image sensor 21 will set the shutter time and the gain relative to the white light synchronously.

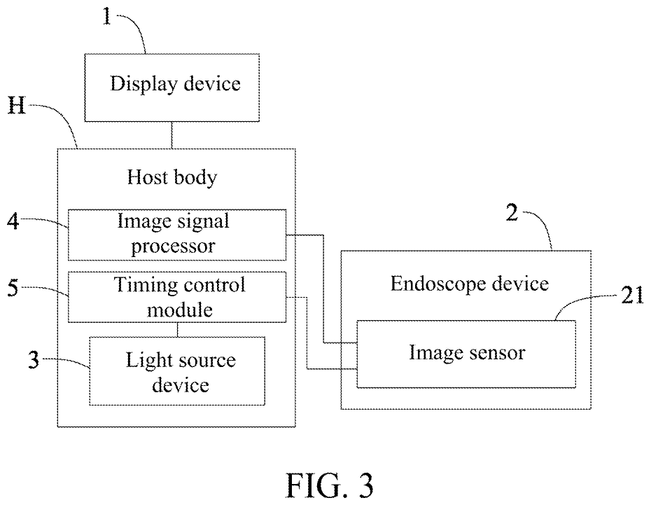

Referring to FIG. 3, FIG. 3 is a block diagram of a second embodiment of an endoscope light source synchronization system of the present disclosure. In this embodiment, the light source device 3 and the timing control module 5 can be provided in the host body H. The light source device 3 may be a combination of a halogen lamp and several filters with different colors; and the light source emitted by the light source device 3 is transmitted to the endoscope device 2 via an optical fiber.

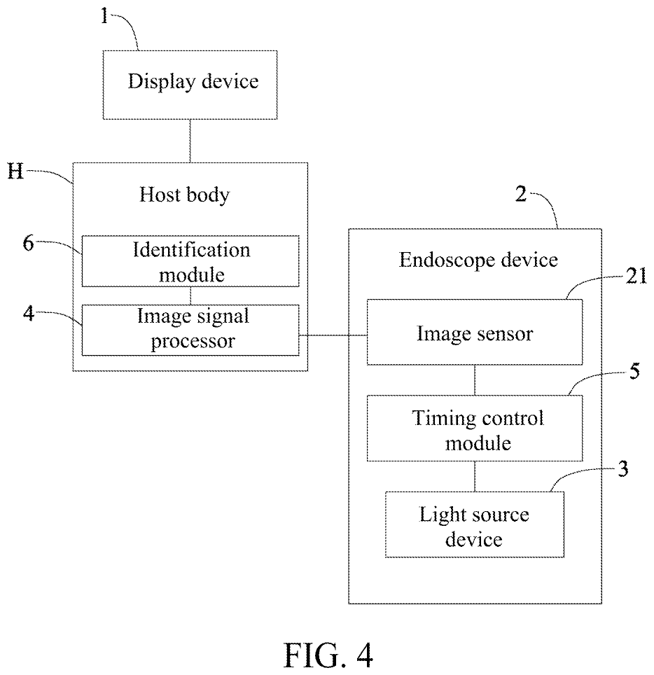

Referring to FIG. 4, FIG. 4 is a block diagram of a third embodiment of an endoscope light source synchronization system of the present disclosure. Compared with the first embodiment, the endoscope light source synchronization system may further include an identification module 6 electrically connected to the image signal processor 4. Specifically, the light sources emitted by the light source device 3 include narrow band imaging light; the identification module 6 performs image identification on image screens generated by the narrow band imaging light to identify whether the part to be detected has a sign or not, and transmits a result to the image signal processor 4. Subsequently, the image signal processor 4 controls the display device 1 to display the result. By way of example, the identification module 6 can be constructed by an artificial neural network such as a deep neural network, a convolutional neural network or a recurrent neural network, which belongs to the general knowledge in the related art of the present disclosure, and will not be repeated here.

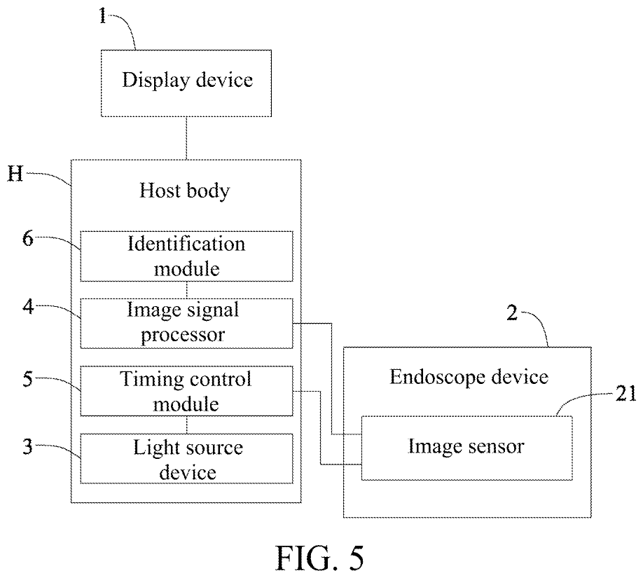

Referring to FIG. 5, FIG. 5 is a block diagram of a fourth embodiment of an endoscope light source synchronization system of the present disclosure. Compared with the second embodiment, the endoscope light source synchronization system may also include the identification module 6.

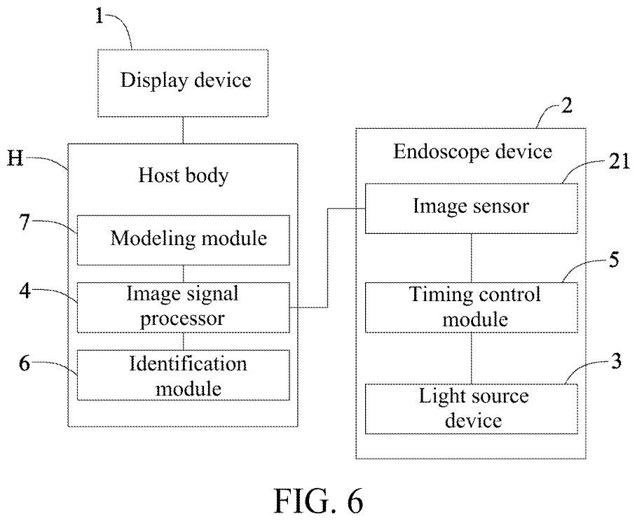

Referring to FIG. 6, FIG. 6 is a block diagram of a fifth embodiment of an endoscope light source synchronization system of the present disclosure. Compared with the third embodiment, the endoscope light source synchronization system may further include a modeling module 7 electrically connected to the image signal processor 4. Specifically, the light sources emitted by the light source device 3 include structured light; the modeling module 7 generates a three-dimensional image according to an image signal generated by the structured light and transmits the three-dimensional image to the image signal processor 4. Subsequently, the image signal processor 4 controls the display device 1 to display the three-dimensional image. By way of example, the modeling module 7 may be related 3D modeling software such as 3ds Max or Unity 3D.

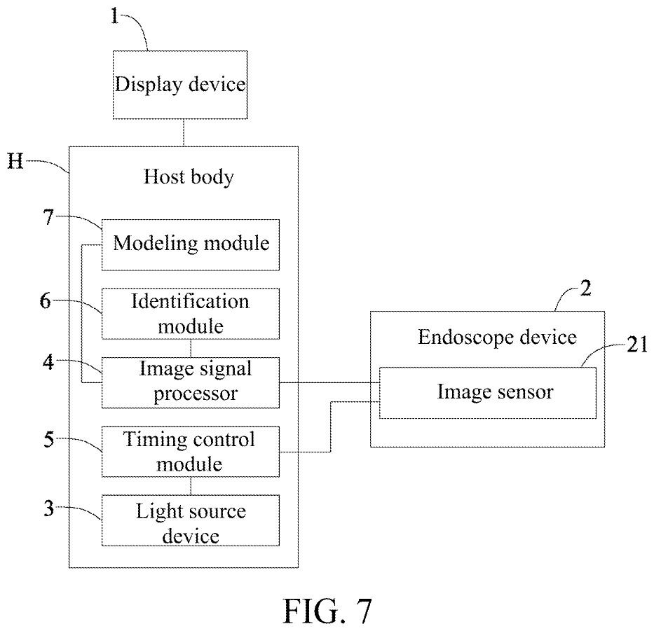

Referring to FIG. 7, FIG. 7 is a block diagram of a sixth embodiment of an endoscope light source synchronization system of the present disclosure. Compared with the fourth embodiment, the endoscope light source synchronization system may also include a modeling module 7. It is worth mentioning that the identification module 6 and the modeling module 7 can both exist simultaneously to display identified results and the three-dimensional images of the part to be detected on the display device 1 simultaneously.

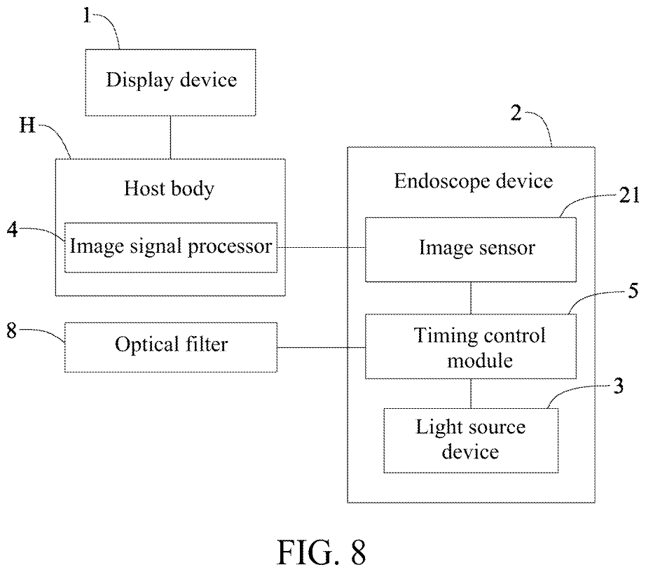

Referring to FIG. 8, FIG. 8 is a block diagram of a seventh embodiment of an endoscope light source synchronization system of the present disclosure. Compared with the first embodiment, the endoscope light source synchronization system may further include an optical filter 8, where the optical filter 8 is provided in front of the image sensor 21 to filter out some wavelengths of light rays. Specifically, the light source device 3 can emit fluorescent excitation light to the part to be detected, so that the part to be detected emits a fluorescence reaction, where the fluorescence reaction includes fluorescent light rays with a specific wavelength. The timing control module 5 uses the synchronization signal to synchronously control the optical filter 8 to switch to an appropriate filter, so that the optical filter 8 receives the fluorescence reaction and filters out a band signal with the fluorescent excitation light in the fluorescence reaction, so as to obtain the fluorescent light rays with a specific wavelength.

By way of example, this embodiment can provide an indocyanine green (ICG) developing function. Specifically, the indocyanine green is a non-radioactive fluorescent dye with a main absorption wavelength of about 720 nm-810 nm. The main fluorescence reaction wavelength emitted after absorption is about 810 nm-900 nm. Therefore, the optical filter 8 that can filter out wavelengths of at least 700 nm-810 nm is selected, and the light source device 3 emits the fluorescent excitation light with a center wavelength between 750 nm-810 nm towards the part to be detected, so that the image sensor 21 can convert the fluorescent light rays with wavelengths between 815 nm-880 nm into corresponding image signals.

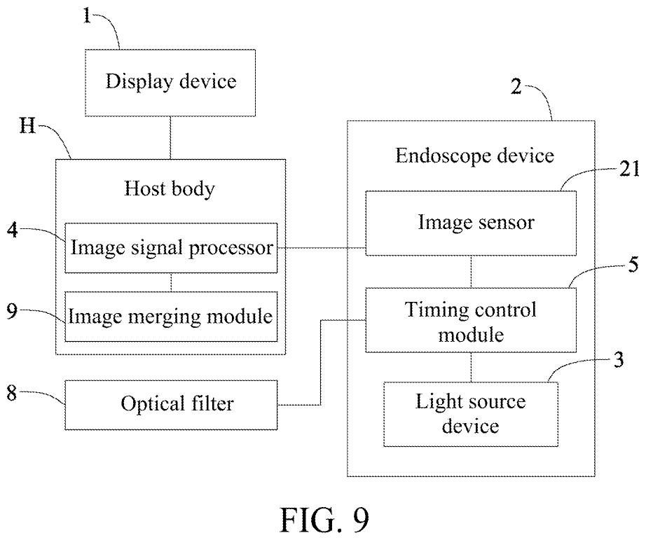

Referring to FIG. 9, FIG. 9 is a block diagram of an eighth embodiment of an endoscope light source synchronization system of the present disclosure. Compared with the seventh embodiment, the endoscope light source synchronization system may further include an image merging module 9, where the image merging module 9 is electrically connected to the image signal processor 4 and is used to merge image screens generated under two different light sources into a fused image.

By way of example, a fused image with fluorescence development can be provided. Specifically, the light source device 3 can emit the white light and the fluorescent excitation light in sequence towards the part to be detected, and after specific wavelengths are filtered out for the fluorescent excitation light with the optical filter 8, the image sensor 21 generates image signals corresponding to the white light and corresponding to the fluorescent excitation light. Subsequently, the image signal processor 4 generates corresponding white light screens and fluorescent screens according to the image signals, and sends the white light screens and the fluorescent screens to the image merging module 9, so that the image merging module merges the white light images with the fluorescent images to form a fused image with fluorescent development.

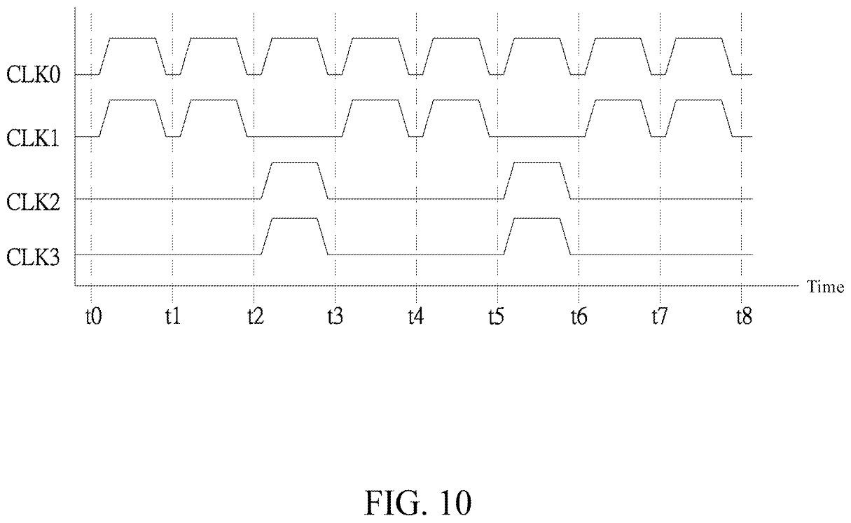

Referring to FIG. 10, FIG. 10 is a timing diagram of a specific embodiment of an endoscope light source synchronization system of the present disclosure, where a 0^{th} frame is a period from t0 to t1, a 1^{st} frame is a period from t1 to t2, a 2^{nd} frame is a period from t2 to t3, a 3^{rd} frame is a period from t3 to t4, a 4^{th} frame is a period from t4 to t5, a 5^{th} frame is a period from t5 to t6, a 6^{th} frame is a period from t6 to t7, and a 7^{th} frame is a period from t7 to t8.

In this embodiment, the light source device 3 includes several light-emitting diodes. The synchronization signal (timing signal CLK0) controls the image sensor 21 to maintain at a high level and continuously operate during the periods from t0 to t8 and the like, and the synchronization signal controls the one (timing signal CLK1) of the several light-emitting diodes that is used to emit the white light to maintain at a high level during the periods from t0 to t2, t3 to t5, t6 to t8 and the like, and maintain at a low level during other periods, and the synchronization signal controls the one (timing signal CLK2) of the several light-emitting diodes that is used to emit the structured light to maintain at a high level during the periods from t2 to t3, t5 to t6 and the like, and maintain at a low level during other periods. Alternatively, the synchronization signal controls the one (timing signal CLK3) of the several light-emitting diodes that is used to emit the narrow band imaging light to maintain at a high level during the periods from t2 to t3, t5 to t6 and the like, and maintain at a low level during other periods. By doing so, the reflected light sensed by the image sensor 21 during the periods from t0 to t2, t3 to t5, t6 to t8 and the like is generated by irradiating the part to be detected with the white light, and the reflected light sensed by the image sensor 21 during the periods from t2 to t3, t5 to t6 and the like is generated by irradiating the part to be detected with the structured light or the narrow band imaging light, so that the image signals corresponding to different light sources such as the white light, the white light, the structured light or the narrow band imaging light, the white light, the white light, the structured light or the narrow band imaging light, the white light, the white light and the like are generated in sequence, and corresponding several image screens are displayed on the display device 1. Since the time for switching the light sources is shorter than the time of persistence of human vision, the image screen relative to the white light and the image screen relative to the structured light or the narrow band imaging light appear to be displayed on the display device 1 simultaneously in the human eye.

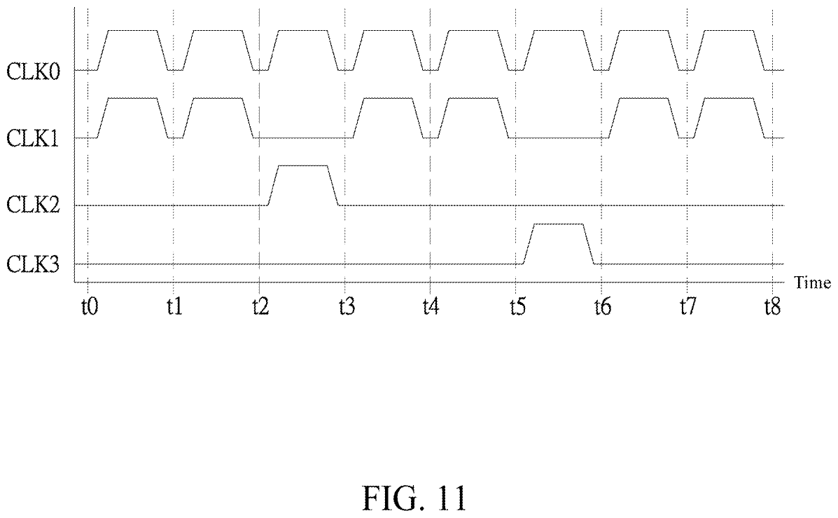

Referring to FIG. 11, FIG. 11 is a timing diagram of another specific embodiment of an endoscope light source synchronization system of the present disclosure, where a 0^{th} frame is a period from t0 to t1, a 1^{st} frame is a period from t1 to t2, a 2^{nd} frame is a period from t2 to t3, a 3^{rd} frame is a period from t3 to t4, a 4^{th} frame is a period from t4 to t5, a 5^{th} frame is a period from t5 to t6, a 6^{th} frame is a period from t6 to t7, and a 7^{th} frame is a period from t7 to t8.

In this embodiment, the light source device 3 includes several light-emitting diodes. The synchronization signal (timing signal CLK0) controls the image sensor 21 to maintain at a high level and continuously operate during the periods from t0 to t8 and the like, and the synchronization signal controls the one (timing signal CLK1) of the several light-emitting diodes that is used to emit the white light to maintain at a high level during the periods from t0 to t2, t3 to t5, t6 to t8 and the like, and maintain at a low level during other periods, and the synchronization signal controls the one (timing signal CLK2) of the several light-emitting diodes that is used to emit the structured light to maintain at a high level during the period from t2 to t3, and maintain at a low level during other periods. Moreover, the synchronization signal controls the one (timing signal CLK3) of the several light-emitting diodes that is used to emit the narrow band imaging light to maintain at a high level during the period from t5 to t6, and maintain at a low level during other periods. By doing so, the reflected light sensed by the image sensor 21 during the periods from t0 to t2, t3 to t5, t6 to t8 and the like is generated by irradiating the part to be detected with the white light, the reflected light sensed by the image sensor 21 during the period from t2 to t3 is generated by irradiating the part to be detected with the structured light, and the reflected light sensed by the image sensor 21 during the period from t5 to t6 is generated by irradiating the part to be detected with the narrow band imaging light, so that the image signals corresponding to different light sources such as the white light, the white light, the structured light, the white light, the white light, the narrow band imaging light, the white light, the white light and the like are generated in sequence, and corresponding several image screens are displayed on the display device 1. Since the time for switching the light sources is shorter than the time of persistence of human vision, the image screen relative to the white light and the image screen relative to the structured light and relative to the narrow band imaging light appear to be displayed on the display device 1 simultaneously in the human eye. In this embodiment, preferably, the endoscope light source synchronization system may be suitable for screens of 30 fps, so that the endoscope light source synchronization system meets specifications of most existing screens, and smooth and low-delay display is implemented while low power consumption is maintained.

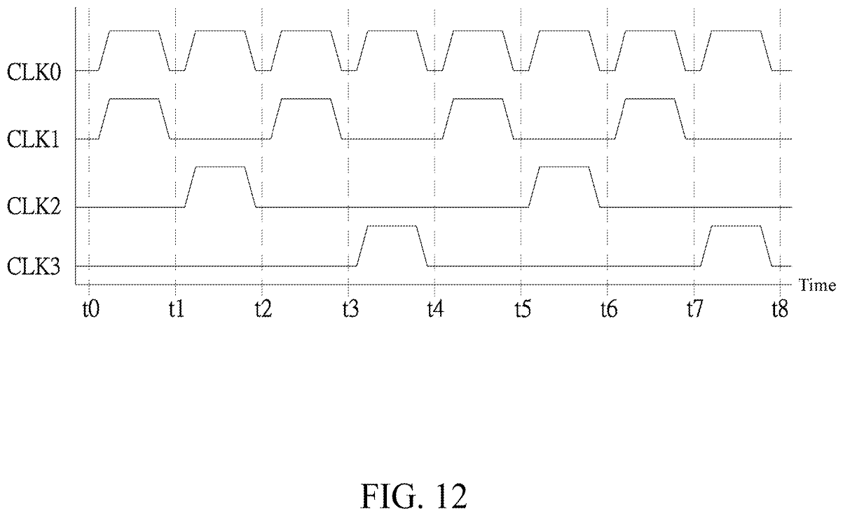

Referring to FIG. 12, FIG. 12 is a timing diagram of yet another specific embodiment of an endoscope light source synchronization system of the present disclosure, where a 0^{th} frame is a period from t0 to t1, a 1^{st} frame is a period from t1 to t2, a 2^{nd} frame is a period from t2 to t3, a 3^{rd} frame is a period from t3 to t4, a 4^{th} frame is a period from t4 to t5, a 5^{th} frame is a period from t5 to t6, a 6^{th} frame is a period from t6 to t7, and a 7^{th} frame is a period from t7 to t8.

In this embodiment, the light source device 3 includes several light-emitting diodes. The synchronization signal (timing signal CLK0) controls the image sensor 21 to maintain at a high level and continuously operate during the periods from t0 to t8 and the like, and the synchronization signal controls the one (timing signal CLK1) of the several light-emitting diodes that is used to emit the white light to maintain at a high level during the periods from t0 to t1, t2 to t3, t4 to t5, t6 to t7 and the like, and maintain at a low level during other periods, and the synchronization signal controls the one (timing signal CLK2) of the several light-emitting diodes that is used to emit the structured light to maintain at a high level during the periods from t1 to t2, t5 to t6 and the like and maintain at a low level during other periods. Moreover, the synchronization signal controls the one (timing signal CLK3) of the several light-emitting diodes that is used to emit the narrow band imaging light to maintain at a high level during the periods from t3 to t4, t7 to t8 and the like and maintain at a low level during other periods. By doing so, the reflected light sensed by the image sensor 21 during the periods from t0 to t1, t2 to t3, t4 to t5, t6 to t7 and the like is generated by irradiating the part to be detected with the white light, the reflected light sensed by the image sensor 21 during the periods from t1 to t2, t5 to t6 and the like is generated by irradiating the part to be detected with the structured light, and the reflected light sensed by the image sensor 21 during the periods from t3 to t4, t7 to t8 and the like is generated by irradiating the part to be detected with the narrow band imaging light, so that the image signals corresponding to different light sources such as the white light, the structured light, the white light, the narrow band imaging light, the white light, the structured light, the white light, the narrow band imaging light and the like are generated in sequence, and corresponding several image screens are displayed on the display device 1. Since the time for switching the light sources is shorter than the time of persistence of human vision, the image screen relative to the white light and the image screen relative to the structured light and relative to the narrow band imaging light appear to be displayed on the display device 1 simultaneously in the human eye. In this embodiment, preferably, the endoscope light source synchronization system may be suitable for screens of 60 fps, so that smoother screens can be displayed on the screen simultaneously, so that when the physician view the images such as the white light image, the structured light image and the narrow band imaging light image through the screen, screen stuttering cannot occur.

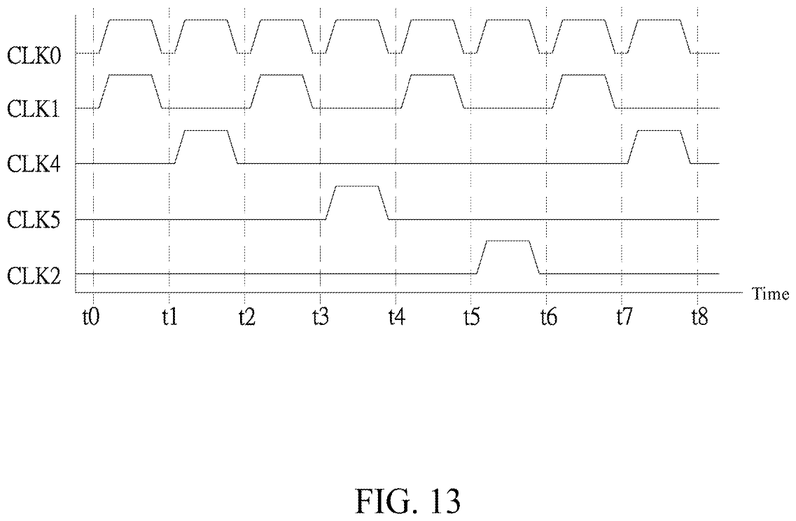

Referring to FIG. 13, FIG. 13 is a timing diagram of still another specific embodiment of an endoscope light source synchronization system of the present disclosure, where a 0^{th} frame is a period from t0 to t1, a 1^{st} frame is a period from t1 to t2, a 2^{nd} frame is a period from t2 to t3, a 3^{rd} frame is a period from t3 to t4, a 4^{th} frame is a period from t4 to t5, a 5^{th} frame is a period from t5 to t6, a 6^{th} frame is a period from t6 to t7, and a 7^{th} frame is a period from T7 to T8.

In this embodiment, the light source device 3 includes several light-emitting diodes. The synchronization signal (timing signal CLK0) controls the image sensor 21 to maintain at a high level and continuously operate during the periods from t0 to t8 and the like, and the synchronization signal controls the one (timing signal CLK1) of the several light-emitting diodes that is used to emit the white light to maintain at a high level during the periods from t0 to t1, t2 to t3, t4 to t5, t6 to t7 and the like, and maintain at a low level during other periods, and the synchronization signal controls the one (timing signal CLK4) of the several light-emitting diodes that is used to emit blue light to maintain at a high level during the periods from t1 to t2, t7 to t8 and the like and maintain at a low level during other periods. Moreover, the synchronization signal controls the one (timing signal CLK5) of the several light-emitting diodes that is used to emit green light to maintain at a high level during the period from t3 to t4 and maintain at a low level during other periods, and the synchronization signal controls the one (timing signal CLK2) of the several light-emitting diodes that is used to emit the structured light to maintain at a high level during the period from t5 to t6, and maintain at a low level during other periods. By doing so, the reflected light sensed by the image sensor 21 during the periods from t0 to t1, t2 to t3, t4 to t5, t6 to t7 and the like is generated by irradiating the part to be detected with the white light, the reflected light sensed by the image sensor 21 during the periods from t1 to t2, t7 to t8 and the like is generated by irradiating the part to be detected with the blue light, the reflected light sensed by the image sensor 21 during the period from t3 to t4 is generated by irradiating the part to be detected with the green light, and the reflected light sensed by the image sensor 21 during the period from t5 to t6 is generated by irradiating the part to be detected with the structured light, so that the image signals corresponding to different light sources such as the white light, the blue light, the white light, the green light, the white light, the structured light, the white light, the blue light and the like are generated in sequence, and corresponding several image screens are displayed on the display device 1. Since the time for switching the light sources is shorter than the time of persistence of human vision, the image screen relative to the white light and the image screens relative to the structured light and relative to the narrow band imaging light generated by the green light and the blue light appear to be displayed on the display device 1 simultaneously in the human eye. In this embodiment, preferably, the endoscope light source synchronization system can be suitable for screens of 120 fps, so that more delicate screens can be displayed on the screen simultaneously, allowing the physician to view whether the part to be detected has the sign or not more clearly.

To sum up, the endoscope light source synchronization system of the present disclosure can synchronously control the image sensor to start operating when the light source device is controlled to start operating, and a set light source irradiates the part to be detected, which can achieve an effect of avoiding that the image screens generated under different light sources skew during switching. Furthermore, in an asynchronous processing way of switching light sources and then shooting images, when the image screens generated under different light sources are displayed on a screen, multiple image screens cannot exist synchronously in human vision, that is, the physician can only view the image screens generated by one of the light sources in the same period. However, when the image screens generated in the present disclosure are displayed on the corresponding screens, respectively, because of the persistence of human vision, the image screens look like a continuous screen when the physician views the image screens on each screen. Therefore, the physician can view the image screens generated by several different light sources respectively in the same period, which achieves an effect of improving the accuracy of the detection and analysis of the physician of whether the part to be detected has the sign or not.

The present disclosure has been disclosed in the preferred embodiments above, but those skilled in the art should understand that these embodiments are only used to illustrate the present disclosure and should not be interpreted as limiting the scope of the present disclosure. It should be noted that all changes and substitutions equivalent to the embodiments should be considered within the scope of the present disclosure. Therefore, the scope of protection of the present disclosure shall be subject to the definition of the scope of claims.

What is claimed is:

1. An endoscope light source synchronization system, adapted to connect a display device,
an endoscope device and a light source device, wherein the endoscope device comprises an image sensor, the light source device is used to emit at least two kinds of light sources and irradiate a part to be detected, and the endoscope light source synchronization system comprises:
an image signal processor, electrically connected to the display device and the image sensor; and
a timing control module, electrically connected to the image sensor and the light source device, wherein the timing control module uses a synchronization signal to synchronously control the light source device and the image sensor, and synchronously controls the light source device to switch and activate the light sources according to different light sources required by the image sensor in each frame, so that the image sensor generates different image signals corresponding to different light sources; and the image signal processor simultaneously displays a plurality of corresponding image screens on the display device according to the image signals.

2. The endoscope light source synchronization system according to claim 1, wherein the light source device comprises a plurality of light-emitting diodes, and the plurality of light-emitting diodes are used to emit light sources with different colors and provided in the endoscope device.

3. The endoscope light source synchronization system according to claim 1, wherein the image signal processor is provided in a host body; an outer surface of the host body is provided with a first connecting hole and a second connecting hole to be electrically connected to the image signal processor respectively; the first connecting hole is used to connect the display device; and the second connecting hole is used to connect the endoscope device.

4. The endoscope light source synchronization system according to claim 3, wherein the light source device is provided in the host body and is a combination of a halogen lamp and a plurality of filters with different colors; and the light source emitted by the light source device is transmitted to the endoscope device via an optical fiber.

5. The endoscope light source synchronization system according to claim 1, wherein the light sources emitted by the light source device are at least two kinds of light sources of white light, structured light, infrared light, ultraviolet light, fluorescent excitation light, and narrow band imaging light.

6. The endoscope light source synchronization system according to claim 3, wherein the timing control module is provided in the endoscope device or the host body.

7. The endoscope light source synchronization system according to claim 1, wherein the image sensor sets a corresponding exposure parameter according to different light sources required in each frame.

8. The endoscope light source synchronization system according to claim 7, wherein the exposure parameters comprise a shutter time and a gain.

9. The endoscope light source synchronization system according to claim 1, further comprising an identification module electrically connected to the image signal processor, wherein the light sources emitted by the light source device comprise narrow band imaging light; the identification module performs image identification on image screens generated by the narrow band imaging light to identify whether the part to be detected has a sign or not, and transmits a result to the image signal processor; and the image signal processor controls the display device to display the result.

10. The endoscope light source synchronization system according to claim 9, further comprising a modeling module electrically connected to the image signal processor, wherein the light sources emitted by the light source device comprise structured light; the modeling module generates a three-dimensional image according to an image signal generated by the structured light and transmits the three-dimensional image to the image signal processor; and the image signal processor controls the display device to display the three-dimensional image.

11. The endoscope light source synchronization system according to claim 1, further comprising an optical filter, wherein the optical filter is provided in front of the image sensor to filter out some wavelengths of light rays.

12. The endoscope light source synchronization system according to claim 11, further comprising an image merging module, wherein the image merging module is electrically connected to the image signal processor and is used to merge image screens generated under two different light sources into a fused image.

13. The endoscope light source synchronization system according to claim 1, further comprising a modeling module electrically connected to the image signal processor, wherein the light sources emitted by the light source device comprise structured light; the modeling module generates a three-dimensional image according to an image signal generated by the structured light and transmits the three-dimensional image to the image signal processor; and the image signal processor controls the display device to display the three-dimensional image.
